(19) United States
(12) Patent Application Publication
(10) Pub. No.: US 2025/0169715 A1
(43) Pub. Date: May 29, 2025

(54) USE OF BLINK REFLEX FOR HEADACHE RESPONSE MONITORING AND/OR TREATMENT SELECTION

(71) Applicant: Blinktbi, Inc., Charleston, SC (US)

(72) Inventors: Douglas CARNES, Charleston, SC (US); Andrew MASSEY, Charleston, SC (US); Jeffrey RILEY, Charleston, SC (US); Jennifer FIORINI, Charleston, SC (US)

(21) Appl. No.: 18/837,441

(22) PCT Filed: Feb. 10, 2023
(86) PCT No.: PCT/US2023/012793

§ 371 Date: Aug. 9, 2024

Related U.S. Application Data
(60) Provisional application No. 63/309,221, filed on Feb. 11, 2022.

(51) Int. Cl. A61B 5/11; A61B 3/113; A61B 5/00; A61K 31/135

(52) U.S. Cl. CPC A61B 5/1103; A61B 3/113; A61B 5/0053; A61B 5/4824; A61B 5/4842; A61B 5/4848; A61B 5/7246; A61K 31/135

(57) ABSTRACT

The disclosure relates to, in part, a method of diagnosing whether a patient is afflicted with a headache and/or selecting headache treatment for a patient afflicted with a headache and/or monitoring response of a headache in patient to a headache treatment.

### CROSS REFERENCE TO RELATED APPLICATIONS

This application claims priority to U.S. Provisional Patent Application No. 63/309,221, filed Feb. 11, 2022, the content of which is hereby incorporated by reference, in its entirety, for all purposes.

### FIELD

The present disclosure provides methods for headache response monitoring and/or treatment selection in a patient.

### BACKGROUND

Headache encompasses a relatively vast spectrum of common symptoms and sometimes obscure causes. Headaches may be associated with emotional states such as depression and tension, as well as physical events such as muscular tension, neuralgia, neuropathy and vascular disturbances. Although a patient may experience more than one kind of headache pain, the various manifestations of headache are generally believed to have different pathological origins. Currently, the tools and methods for diagnosing whether a patient is afflicted with headache are unreliable. There is a need for effective methods of diagnosing, predicting, and/or treating headache in a patient. Moreover, there is a need for effective methods of selecting treatment and/or monitoring response to treatment in patient afflicted with a migraine disorder.

### SUMMARY

Accordingly, the present disclosure provides a method of diagnosing whether a patient is afflicted with a headache, comprising: (a) measuring a blink reflex in a patient suspected to be afflicted with a headache, the measurement comprising: (i) applying a non-electrical stimulus to an eye of the patient to induce a blink reflex and (ii) assessing one or more blink-associated parameters; and (b) diagnosing the patient as afflicted with a headache or not afflicted with a headache by comparing the one or more blink-associated parameters to equivalent one or more blink-associated parameters from a patient who is not afflicted with a headache.

In aspects, the present disclosure provides a method of predicting the onset of a headache in a patient afflicted with a headache, comprising: (a) measuring a blink reflex in the patient, the measurement comprising: (i) applying a non-electrical stimulus to an eye of the patient to induce a blink reflex and (ii) assessing one or more blink-associated parameters; and (b) determining if the patient is likely to experience an onset of a headache by comparing the one or more blink-associated parameters to equivalent one or more blink-associated parameters from a patient who is not afflicted with a headache and/or the patient when not experiencing symptoms of a headache or experiencing the onset of a headache.

In embodiments, the present disclosure provides a method of diagnosing and treating a patient afflicted with a headache, comprising: (a) measuring a blink reflex in a patient suspected to be afflicted with a headache, the measurement comprising: (i) applying a non-electrical stimulus to an eye of the patient to induce a blink reflex and (ii) assessing one or more blink-associated parameters; (b) diagnosing the patient as afflicted with a headache or not afflicted with a headache by comparing the one or more blink-associated parameters to equivalent one or more blink-associated parameters from a patient who is not afflicted with a headache; and (c) administering an effective amount of a headache treatment to a patient diagnosed as afflicted with a headache.

In aspects, the present disclosure provides a method of selecting headache treatment for a patient afflicted with a headache, comprising: (a) measuring a blink reflex in the patient afflicted with a headache, the measurement comprising: (i) applying a non-electrical stimulus to an eye of the patient to induce a blink reflex and (ii) assessing one or more blink-associated parameters; (b) selecting a headache treatment for the patient afflicted with a headache by comparing the one or more blink-associated parameters to equivalent one or more blink-associated parameters from the patient when not experiencing symptoms of a headache and/or the patient when receiving the headache treatment.

In embodiments, the present disclosure provides a method of monitoring response of a headache in patient to a headache treatment, comprising (a) measuring a blink reflex in the patient afflicted with a headache and who has undergone or is undergoing treatment with the headache treatment, the measurement comprising: (i) applying a non-electrical stimulus to an eye of the patient to induce a blink reflex and (ii) assessing one or more blink-associated parameters; (b) determining a response to the headache treatment by comparing the one or more blink-associated parameters to equivalent one or more blink-associated parameters from the patient when not experiencing symptoms of a headache and/or the patient before receiving the headache treatment.

In embodiments, the headache is or comprises a migraine disorder.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1A-B is a non-limiting schematic representation of the blink reflex biomarker. The blink reflex is a physiological biomarker of neurological function involving two cranial nerves, the trigeminal (V) and facial (VII) nerves, routing through the medulla and pons in the brainstem. FIG. 1A is a non-limiting schematic representation of a patient undergoing measurement of the blink reflex with a blink reflex monitoring device. FIG. 1B is a non-limiting schematic representation of a applying a non-electrical stimulus (e.g., without limitation, a puff of air, light, heat, and/or sound) to the canthus or outer canthus of the patient, which sends a signal through the trigeminal (V) and facial (VII) nerves, routing through the medulla and pons in the brainstem, and resulting in a blink reflex that is measured with a device (e.g., without limitation, a high-speed camera).

FIG. 2 shows Pre & Post Treatment Pain Scores. A visual analog scale (VAS) was used to assess pain scores from patients with headache/migraine before and after undergoing all treatment modalities for headache/migraine pain.

FIG. 3A-D shows Pre & Post Reflex Variables by Treatment Modality. Multiple blink-associated parameters from patients with headache/migraine before and after undergoing different treatments were analyzed. The different treatments were intravenous (IV) ketamine infusion therapy, sphenopalatine ganglion (SPG) block+peripheral nerve block (PNB), and IV ketamine+SPG+PNB. Significant p=<0.05. FIG. 3A depicts latency. FIG. 3B depicts delta 30. FIG. 3C depicts number of oscillations. FIG. 3D depicts number of blinks.

FIG. 4A-B shows Pre & Post Reflex Variables by Treatment Modality. Multiple blink-associated parameters from patients with headache/migraine before and after undergoing different treatments were analyzed. The different treatments were intravenous (IV) ketamine infusion therapy, sphenopalatine ganglion (SPG) block+peripheral nerve block (PNB), and IV ketamine+SPG+PNB. Significant p=<0.05. FIG. 4A depicts blink rate. FIG. 4B depicts maximum opening velocity.

FIG. 5A-D shows Pre & Post Reflex Variables by Treatment Modality. Multiple blink-associated parameters from patients with headache/migraine before and after undergoing different treatments were analyzed. The different treatments were intravenous (IV) ketamine infusion therapy, sphenopalatine ganglion (SPG) block+peripheral nerve block (PNB), and IV ketamine+SPG+PNB. non-significant p=>0.05. FIG. 5A depicts differential latency. FIG. 5B depicts time under 20. FIG. 5C depicts initial lid velocity. FIG. 5D depicts time to close.

FIG. 6A-D shows Pre & Post Reflex Variables by Treatment Modality. Multiple blink-associated parameters from patients with headache/migraine before and after undergoing different treatments were analyzed. The different treatments were intravenous (IV) ketamine infusion therapy, sphenopalatine ganglion (SPG) block+peripheral nerve block (PNB), and IV ketamine+SPG+PNB. non-significant p=>0.05. FIG. 6A depicts time to open. FIG. 6B depicts area under curve. FIG. 6C depicts maximum closing velocity. FIG. 6D depicts excursion.

FIG. 7 shows time to first oscillation from Pre & Post Reflex Variables by Treatment Modality. Time to first oscillation, which is one blink-associated parameter, from patients with headache/migraine before and after undergoing different treatments was analyzed. The different treatments were intravenous (IV) ketamine infusion therapy, sphenopalatine ganglion (SPG) block+peripheral nerve block (PNB), and IV ketamine+SPG+PNB. non-significant p=>0.05.

FIG. 8A-C shows Pre & Post Treatment VAS Pain Score by Treatment Modality. A visual analog scale (VAS) was used to assess pain scores from patients before and after undergoing each treatment modalities for headache/migraine pain. The different treatments were intravenous (IV) ketamine infusion therapy, sphenopalatine ganglion (SPG) block+peripheral nerve block (PNB), and IV+SPG+PNB. FIG. 8A depicts treatment with IV. FIG. 8B depicts treatment with SPG+PNB. FIG. 8C depicts treatment with IV ketamine+SPG+PNB.

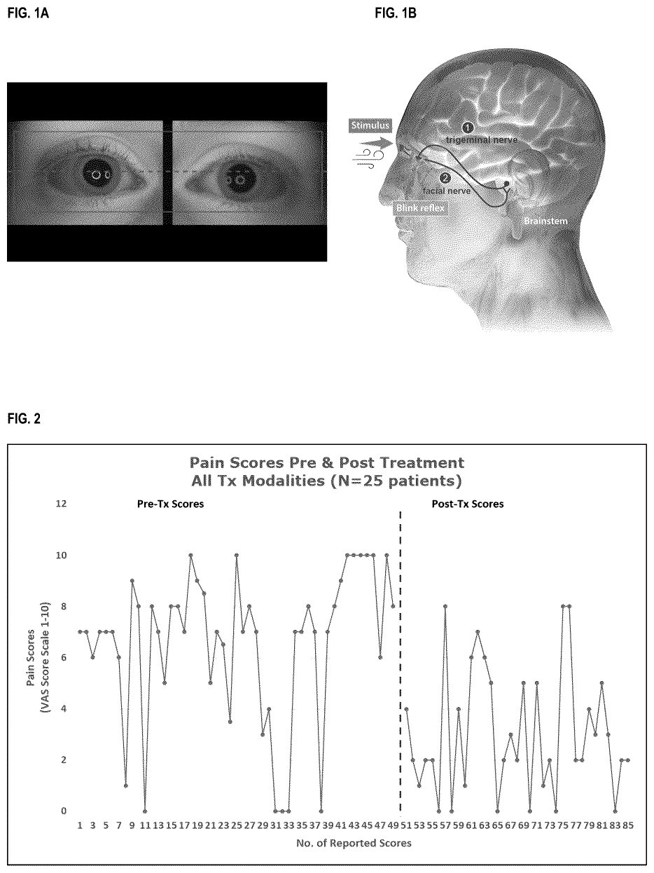

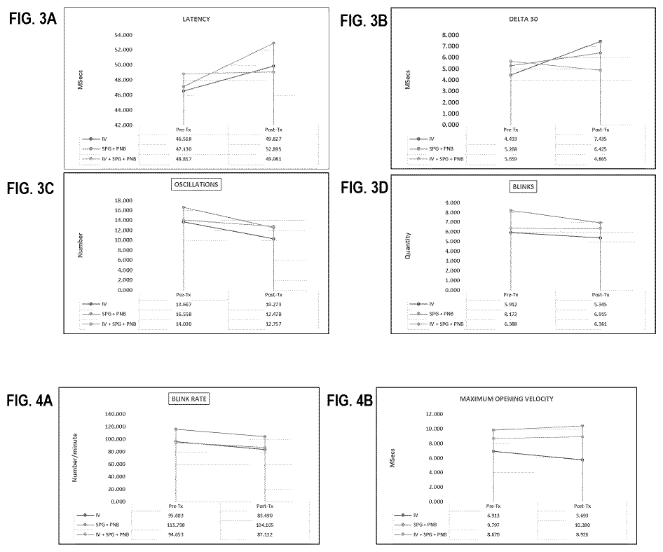

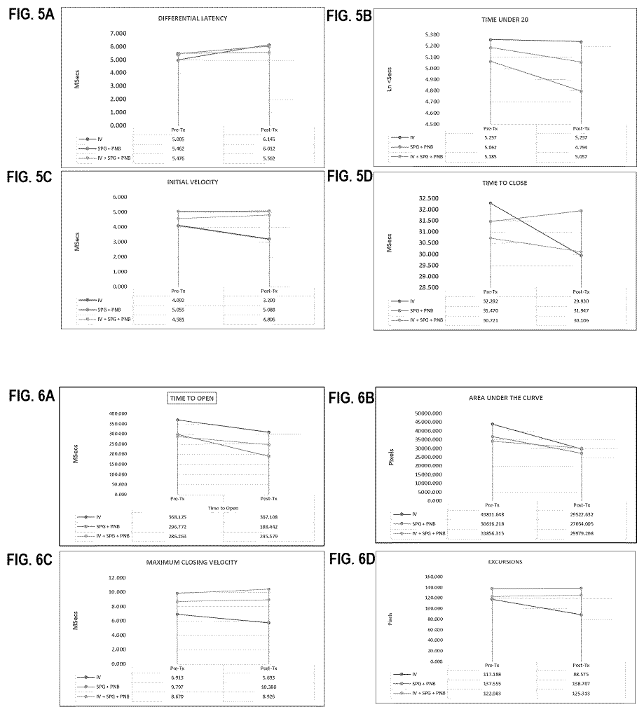

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

The present disclosure provides a method of diagnosing whether a patient is afflicted with a headache, comprising: (a) measuring a blink reflex in a patient suspected to be afflicted with a headache, the measurement comprising: (i) applying a non-electrical stimulus to an eye of the patient to induce a blink reflex and (ii) assessing one or more blink-associated parameters; and (b) diagnosing the patient as afflicted with a headache or not afflicted with a headache by comparing the one or more blink-associated parameters to equivalent one or more blink-associated parameters from a patient who is not afflicted a with headache.

In aspects, the present disclosure provides a method of predicting the onset of a headache in a patient afflicted with a headache, comprising: (a) measuring a blink reflex in the patient, the measurement comprising: (i) applying a non-electrical stimulus to an eye of the patient to induce a blink reflex and (ii) assessing one or more blink-associated parameters; and (b) determining if the patient is likely to experience an onset of a headache by comparing the one or more blink-associated parameters to equivalent one or more blink-associated parameters from a patient who is not afflicted with a headache and/or the patient when not experiencing symptoms of a headache or experiencing the onset of a headache.

In embodiments, the methods of the present disclosure provide information that can be used for pre-emptive treatment to avoid the onset of a headache. In embodiments, the methods of the present disclosure provide relevant information for preventing further development of a headache. In embodiments, the methods of the present disclosure provide relevant information for preventing development of an attack stage of a headache.

In embodiments, the present disclosure provides a method of diagnosing and treating a patient afflicted with a headache, comprising: (a) measuring a blink reflex in a patient suspected to be afflicted with a headache, the measurement comprising: (i) applying a non-electrical stimulus to an eye of the patient to induce a blink reflex and (ii) assessing one or more blink-associated parameters; and (b) diagnosing the patient as afflicted with a headache or not afflicted with a headache by comparing the one or more blink-associated parameters to equivalent one or more blink-associated parameters from a patient who is not afflicted with a headache; and (c) administering an effective amount of a headache treatment to a patient diagnosed as afflicted with a headache.

In aspects, the present disclosure provides a method of selecting headache treatment for a patient afflicted with a headache, comprising: (a) measuring a blink reflex in the patient afflicted with a headache, the measurement comprising: (i) applying a non-electrical stimulus to an eye of the patient to induce a blink reflex and (ii) assessing one or more blink-associated parameters; and (b) selecting a headache treatment for the patient afflicted with a headache by comparing the one or more blink-associated parameters to equivalent one or more blink-associated parameters from the patient when not experiencing symptoms of a headache and/or the patient when receiving the headache treatment.

In embodiments, the present disclosure provides a method of monitoring response of a headache in patient to a headache treatment, comprising (a) measuring a blink reflex in the patient afflicted with a headache and who has undergone or is undergoing treatment with the headache treatment, the measurement comprising: (i) applying a non-electrical stimulus to an eye of the patient to induce a blink reflex and (ii) assessing one or more blink-associated parameters; and (b) determining a response to the headache treatment by comparing the one or more blink-associated parameters to equivalent one or more blink-associated parameters from the patient when not experiencing symptoms of a headache and/or the patient before receiving the headache treatment.

In embodiments, the methods of the present disclosure provide information that may inform modifying the patient's treatment (e.g., without limitation, changing treatment (e.g., adding or removing one or more headache treatments), altering dosage of the headache treatment(s), and/or altering the headache treatment regimen (e.g., altering frequency and/or duration of dosing).

In embodiments, the diagnosing comprises: diagnosis of disease onset; determination of disease stage and/or severity; determination of disease progress; and/or determination of cognitive state and/or mental capacity.

In embodiments, the method is carried out remotely, optionally with a telehealth device.

In embodiments, the method is adapted for point-of-care application.

In embodiments, an information collected can predict correlation of ADHD to the occurrence of a future disease and/or disorder of the central nervous system.

In embodiments, an information collected further allows for assessing the therapeutic efficacy of pharmaceutical and/or medical and/or surgical treatment modalities.

In embodiments, the method of the present disclosure measures latency until physiological response to stimulus. In embodiments, the blink reflex correlates with functionality of one or more of the trigeminal and facial nerves.

In embodiments, the stimulus is non-electrical. In embodiments, the non-electrical stimulus is one or more physical stimuli. In embodiments, the one or more physical stimuli are mechanical, visual, and/or acoustic. In embodiments, the one or more physical stimuli are light, heat, and/or sound. In embodiments, the one or more physical stimuli are a puff of air, a flash of light, a noise, and/or a vibration. In embodiments, wherein the stimulus is a sound, the stimulus triggers blink reflex when the sound is about 40 to about 60 dB greater than the minimum dB level for the patient. In embodiments, the puff of air is a short light gust of air. In embodiments, the puff of air is a tactile stimulus. In embodiments, the air is or comprises compressed air. In embodiments, the compressed air is delivered to the user's and/or patient's eye at a predetermined pressure. In embodiments, the puff of air has a pressure of about 3.75 psi (˜25 kPa) to about 60 psi˜415 kPa) (e.g., about 5 kPa, or about 10 kPa, or about 15 kPa, or about 20 kPa, or about 25 kPa). In embodiments, the puff of air is delivered at a duration of about 100 ms and a peak pressure of about 60 psi (˜415 kPa). In embodiments, the air is or comprises carbon dioxide. In embodiments, the air is or comprises nitrogen. In embodiments, the air is or comprises oxygen. In embodiments, the air puff stimulus is delivered from an air source. In embodiments, air source comprises an air compressor. In embodiments, air source comprises an air compression system. In embodiments, air source comprises a tank of industrial air. In embodiments, the air is purified air, usually food grade versions of air, CO₂, nitrogen, etc, are used.

In embodiments, the air compression system may include a gas source, holding tank, power source, and regulator. In embodiments, the holding tank may be coupled to gas source and may receive gas outputted from gas source. In embodiments, the regulator controls gas source and outputs gas from gas source into holding tank prior to being release to the user or patient to initiate a blink reflex. In embodiments, the regulator may be a valve and may be coupled to power source and gas source. In embodiments, the regulator may be coupled to gas source or may be integrally formed with gas source. In embodiments, the regulator may be configured to reduce the pressure of the gas outputted by gas source. For example, the regulator may be configured to step down the pressure outputted from gas source to under 100 PSI. In embodiments, the pressure of the gas outputted by gas source is approximately 900 PSI and regulator is configured to step down the pressure from gas source to less than 100 PSI. However, the regulator may be configured to step down the pressure to less than about 500 PSI, less than about 400 PSI, less than about 200 PSI, or less than about 50 PSI. In embodiments, the regulator is configured to allow certain amounts of pressure into holding tank that can then be released at different pressures to initiate one or more blink reflexes. In embodiments, the regulator may be configured to allow for different outputs of pressure of gas for different users or patients of blink reflex device.

In embodiments, the regulator may be configured to fine tune the pressure of gas outputted by blink reflex that initiates the blink reflex of the user or patient. In embodiments, the regulator may be configured to output gas (via gas source) at the minimum pressure required to initiate a blink reflex. For example, regulator may be configured to output gas at a pressure of less than about 50 PSI. In embodiments, the regulator is configured to received compressed gas from gas source and output compressed gas from blink reflex device at a predetermined pressure for a predetermined duration. For example, the regulator may be configured to output compressed gas at a pressure of about 3 PSI to about 60 PSI or at a pressure of about 30 PSI to about 50 PSI. However, regulator may be configured to output gas at a pressure of about 10 PSI to about 100 PSI, about 20 PSI to about 80 PSI, or about 40 PSI to about 60 PSI. In embodiments, regulator outputs a first puff of compressed gas at a first pressure and then a second puff of compressed air at a second pressure. In embodiments, the first pressure may be less than the second pressure. In embodiments, the regulator outputs compressed gas at the first pressure for one or more puffs and then outputs compressed gas at the second pressure for one or more puffs.

In embodiments, the regulator is to be configured to determine the minimum pressure required to initiate a blink reflex. During use, the regulator may initially output compressed gas at a low pressure and gradually increase the pressure of compressed gas outputted until a blink reflex is detected. In embodiments, the regulator increases the pressure of compressed gas by about 1 to about 3 PSI. However, regulator may increase the pressure of compressed gas by about 0.5 PSI to about 5 PSI, about 1 PSI to about 3 PSI, or less than about 0.5 PSI.

In embodiments, the stimulus is delivered to the desired location, e.g., without limitation, on the face of the subject. In embodiments, the stimulus is delivered to the forehead. In embodiments, the stimulus is delivered to the cheek. In embodiments, the compressed air is applied across the left eye, the right eye, and/or both eyes. In embodiments, the compressed air is applied across the eye. In embodiments, the non-electrical stimulus is applied to the canthus, outer canthus, inner canthus, lateral canthus, or medial canthus. In embodiments, applying the non-electrical stimulus to the canthus or the outer canthus produces a robust blink reflex. In embodiments, the non-electrical stimulus is not applied directly onto the cornea.

In embodiments, the blink reflex is measured with a high-speed camera. In embodiments, the blink reflex is measured by capturing and analyzing high-definition images. In embodiments, the present measurement, e.g., via a device for measuring, capturing, and analyzing high-definition images is further capable of distinguishing between the desired blink and undesired blink. In embodiments, the desired blink is triggered as a result of a blink reflex. In embodiments, the desired blink is triggered by a stimulus (e.g., without limitation, a puff of air, light, heat, and/or sound). In embodiments, the undesired blink is a false-positive blink, a false-negative blink, or an abnormal blink. In embodiments, the desired blink is or comprises an induced blink. In embodiments, the undesired blink is or comprises spontaneous and/or voluntary blink. In embodiments, the present measurement, e.g., via a device for measuring, capturing, and analyzing high-definition images, omits or flags the undesired blink (e.g., without limitation, a false-positive blink, a false-negative blink, or an abnormal blink) from the final results generated. In embodiments, the present measurement, e.g., via a device for measuring, capturing, and analyzing high-definition images, includes the undesired blink (e.g., without limitation, a spontaneous and/or voluntary blink) in the final results generated. In embodiments, the present measurement, e.g., via a device for measuring, capturing, and analyzing high-definition images, includes the desired blink (e.g., without limitation, an induced blink) in the final results generated.

In embodiments, the blink reflex is not measured with an EMG sensor. In embodiments, the blink reflex is measured with a blink reflex monitoring device. In embodiments, the blink reflex monitoring device is a wearable device or a handheld device or can be placed on a tripod, on a desk, or the like. In embodiments, the blink reflex monitoring device is simply placed on a stool or a chair. In embodiments, the blink reflex monitoring device is simply hung on a wall such that the device is able to monitor and record the blink reflex of the user's and/or patient's eye. In embodiments, the blink reflex monitoring device is a wearable device. In embodiments, the blink reflex monitoring device can be worn by a user and/or patient. In embodiments, the blink reflex monitoring device is secured to a user's and/or patient's head via one or more straps. In embodiments, the blink reflex monitoring device is portable. In embodiments, the blink reflex monitoring device may be configured to be disposed on a mount or stand to easily allow for consecutive uses by multiple users. In embodiments, securing the blink reflex monitoring device to a tripod or stand allows for multiple users or patients to each quickly and efficiently use the device without having to constantly remove the device from a user's or patient's head.

In embodiments, the blink reflex monitoring device has or comprises:
a front end, a back end, a first plane extending through the front end and the back end,
a second plane intersecting the first plane between the front end and the back end, the back end configured to receive a portion of a face of a user and disposed opposite the front end; and
a strap coupled to the blink reflex monitoring device and having a first position and a second position,
   wherein in the first position the strap extends away from the blink reflex monitoring device along the first plane end and is configured to secure the blink reflex monitoring device to a head of a user and in the second position the strap extends below the blink reflex monitoring device along the second plane and is configured to support the blink reflex monitoring device above a surface.

In embodiments, the blink reflex monitoring device has or comprises:
a front end and a back end, the back end configured to receive a portion of a face of a user and disposed opposite the front end;
at least one strap coupled to the blink reflex monitoring device proximate the back end, the at least one strap configured to be secured around a head of a user; and
an air compression system including a source of compressed air and a valve, the air compression system disposed within the blink reflex monitoring device and the valve configured to output compressed air from the back end at a pressure of about 3 PSI to about 60 PSI.

In embodiments, the air compression system disposed within the blink reflex monitoring device and the valve configured to output compressed air from the back end at a pressure of about 30 PSI to about 50 PSI.

In embodiments, the blink reflex is not measured with an electromyography (EMG) device. In embodiments, the blink reflex is measured with a high-speed camera. In embodiments, the blink reflex is not measured with an EMG sensor.

In embodiments, the measuring of a blink reflex, comprises:
providing a blink reflex monitoring device to a user, the blink reflex monitoring device having an air compression system including a compressed air source and a valve;
outputting a first burst of compressed air from the compressed air source through the valve at a first pressure;
detecting a presence or an absence of a blink in response to the first burst of compressed air source at the first pressure;
if the blink is present, then measuring a duration between the output of the first burst of compressed air at the first pressure and the blink to determine the blink reflex; and
if the blink is absent, then outputting a second burst of compressed air from the compressed air source at a second pressure, the second pressure being greater than the first pressure and measuring a duration between the output of the second burst of compressed air at the second pressure and the blink to determine the blink reflex.

In embodiments, the method of the present disclosure can detect a blink reflex. In embodiments, the method of the present disclosure can detect a blink reflex and distinguish the blink reflex from a false-positive or a false-negative blink. In embodiments, the method of the present disclosure can detect a blink reflex and distinguish the blink reflex from an abnormal blink. In embodiments, the abnormal blink includes double blink, excessive blink, blinking during micro-sleep, and the like. Abnormal blink may occur when one or both eyes transition from the open state to the close state and begin returning to the open state at rate that is substantially slower than that associated with a normal blink. Such abnormal blink may be an indication of the user or patient experiencing fatigue and/or may occur over a period of that that is substantially longer than the normal blink reflex (e.g., without limitation, 5 times longer, 10 times longer, or 20 times longer). In embodiments, the method of the present disclosure omits or flags the abnormal blink from the final results generated.

In a non-limiting embodiment, the one or more blink-associated parameters are measured in one or more of the right and left eyes.

In embodiments, the one or more blink-associated parameters are selected from:
(a) latency, optionally in milliseconds, comprising a time differential between stimulation and eyelid movement, e.g., one or more of upper eyelid or lower eyelid;
(b) differential latency, optionally in milliseconds, comprising a time differential between the start of ipsilateral eye movement and the start of contralateral eye movement;
(c) delta 30, comprising a time difference between ipsilateral eye and contralateral eye movement;
(d) eyelid excursion, optionally in pixels, comprising a distance traveled by the eyelid from the tonic lid position to closed position;
(e) initial lid velocity, optionally in pixels/msec, comprising an average eyelid speed following start of eyelid movement, e.g., the first about 5 frames, the first about 7 frames, or the first about 10 frames;
(f) time to close, optionally in log scale, comprising a time for lid to travel from tonic lid position to the closed position;
(g) time to open, optionally in log scale, comprising a time for lid to travel from closed position back to tonic lid position;
(h) time under threshold, optionally in log scale, comprising a time that the eyelid spends below the threshold position;
(i) number of oscillations, comprising cycles of up and down upper eyelid movement after a stimulated blink;
(j) total blink time, optionally in log scale, comprising a time from start of eyelid movement until it returns to its tonic lid position;
(k) number of blinks;
(l) blink rate, optionally per minute;
(m) area under curve, optionally in pixels;
(n) maximum closing velocity, optionally in milliseconds, comprising a maximum velocity during eyelid closure;
(o) maximum opening velocity, optionally in milliseconds, comprising a maximum velocity during eyelid opening; and
(p) time to first oscillation, optionally in log scale, comprising the elapsed time between the eyelid returning within threshold of a tonic position for a stimulated blink and the onset of the next unstimulated blink.

In a non-limiting embodiment, the one or more blink-associated parameters are measured in one or more of the right and left eyes.

In embodiments, in comparison of the patient before receiving the headache treatment to after receiving the headache treatment
latency increases;
differential latency increases;
delta 30 increases;
eyelid excursion decreases;
initial lid velocity decreases;
time to close decreases;
time to open decreases;
time under threshold decreases;
number of oscillations decreases;
total blink time decreases;
number of blinks decreases;
blink rate decreases;
area under curve decreases;
maximum closing velocity decreases;
maximum opening velocity decreases; and/or
time to first oscillation increases.

In embodiments, by “increase” or “decreases”, e.g., in comparison of the one or more blink-associated parameters from a patient before receiving the headache treatment to after receiving the headache treatment, the disclosure refers to values that change over time or relative to each other. In embodiments “increase” is synonymous with “is more than” and/or reflects a non-static value that has changed. In embodiments “decrease” is synonymous with “is less than” and/or reflects a non-static value that has changed. In embodiments, assessment of whether a value “increases” or “is more than” or “decreases” or “is less than”, e.g., in comparison of the one or more blink-associated parameters from a patient before receiving the headache treatment to after receiving the headache treatment, involves comparing a measured value at two different time points.

In embodiments, in comparison of the patient before receiving the headache treatment to after receiving the headache treatment delta 30 increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of the patient before receiving the headache treatment to after receiving the headache treatment eyelid excursion decreases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment initial lid velocity decreases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment time to close decreases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment time to open decreases by about or at least about 5%, or by about or at least about 10%, or by about or at least about 15%, or by about or at least about 20%, or by about or at least about 25%.

In embodiments, in comparison of the patient before receiving the headache treatment to after receiving the headache treatment time under threshold of 20 decreases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment number of oscillations decreases by about or at least about 5%, or by about or at least about 10%, or about or by at least about 15%, or by about or at least about 20% %, or by about or at least about 25%.

In embodiments, in comparison of the patient before receiving the headache treatment to after receiving the headache treatment number of blinks decreases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment blink rate decreases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of the patient before receiving the headache treatment to after receiving the headache treatment area under curve decreases by about or at least about 5%, or by about or at least about 10%, or by about or at least about 15%, or by about or at least about 20%, by about or at least about 25%, or by about or at least about 30%, or by about or at least about 35%, or by about or at least about 40%, by about or at least about 45%, or by about or at least about 50%.

In embodiments, in comparison of the patient before receiving the headache treatment to after receiving the headache treatment maximum closing velocity decreases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment maximum opening velocity decreases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the headache treatment to after receiving the headache treatment time to first oscillation increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In a non-limiting embodiment, the one or more blink-associated parameters are measured in one or more of the right and left eyes.

In embodiments, in comparison of a patient who is not afflicted with a headache with a patient who is afflicted with a headache:
latency decreases;
differential latency decreases;
delta 30 decreases;
eyelid excursion increases;
initial lid velocity increases;
time to close increases;
time to open decreases;
time under threshold increases;
number of oscillations increases;
total blink time decreases;
number of blinks increases;
blink rate increases;
area under curve increases;
maximum closing velocity increases; and/or
maximum opening velocity increases.

In embodiments, by “increase” or “decreases”, e.g., in comparison of a patient who is not afflicted with a headache with a patient who is afflicted with a headache, the one or more blink-associated parameters do not change but one or more are static values that are compared to each other. In embodiments “increase” is synonymous with “is more than”. In embodiments “decrease” is synonymous with “is less than”. In embodiments, assessment of whether a value “increases” or “is more than” or “decreases” or “is less than”, e.g., in comparison of a patient who is not afflicted with a headache with a patient who is afflicted with a headache, involves comparing a measured value, e.g., from a patient who is afflicted with a headache, with a references standard, e.g., from a patient or population of patients who are not afflicted with a headache. Accordingly, in embodiments, the one or more blink-associated parameters of a patient or population of patients who is not afflicted with a headache are a reference value.

In embodiments, in comparison of a patient who is not afflicted with a headache with a patient who is afflicted with a headache delta 30 decreases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%, or by about or at least about 55%, or by about or at least about 60%, or by about or at least about 65%, or by about or at least about 70%, or by about or at least about 75%, or by about or at least about 80%, or by about or at least about 85%, or by about or at least about 90%, or by about or at least about 95%, or by about or at least about 100%.

In embodiments, in comparison of a patient who is not afflicted with a headache with a patient who is afflicted with a headache eyelid excursion increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of a patient who is not afflicted with a headache with a patient who is afflicted with a headache initial lid velocity increases by about or at least about 5%, or by about or at least about 10%, or by about or at least about 15%, or by about or at least about 20%, or by about or at least about 25%.

In embodiments, in comparison of a patient who is not afflicted with a headache with a patient who is afflicted with a headache time to close increases by about or at least about 5%, or by about or at least about 10%, or by about or at least about 15%, or by about or at least about 20%, or by about or at least about 25%.

In embodiments, in comparison of a patient who is not afflicted with a headache with a patient who is afflicted with a headache time to open decreases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of a patient who is not afflicted with a headache with a patient who is afflicted with a headache time under threshold of 20 increases by about or at least about 0%, or by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of a patient who is not afflicted with a headache with a patient who is afflicted with a headache number of oscillations increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of a patient who is not afflicted with a headache with a patient who is afflicted with a headache number of blinks increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of a patient who is not afflicted with a headache with a patient who is afflicted with a headache blink rate increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of a patient who is not afflicted with a headache with a patient who is afflicted with a headache area under curve increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of a patient who is not afflicted with a headache with a patient who is afflicted with a headache maximum closing velocity increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of a patient who is not afflicted with a headache with a patient who is afflicted with a headache maximum opening velocity increases by about or at least about 10%, or by about or at least about 20%, or by about or at least about 30%, or by about or at least about 40%, or by about or at least about 50%, or by about or at least about 60%, or by about or at least about 70%, or by about or at least about 80%, or by about or at least about 90%, or by about or at least about 100%.

In embodiments, the headache treatment is one or more of a medical (e.g., pharmaceutical) and surgical treatment.

In embodiments, the headache treatment is selected from over-the-counter medications such as aspirin, ibuprofen (e.g., Advil and Motrin IB), and acetaminophen (e.g., Tylenol), prescription medications, such as triptans such as sumatriptan (e.g., Imitrex) and zolmitriptan (e.g., Zomig), and preventive medications such as metoprolol (e.g., Lopressor), propranolol (e.g., Innopran and Inderal), amitriptyline, divalproex (e.g., Depakote), topiramate (e.g., Qudexy XR, Trokendi XR, and Topamax), and erenumab-aooe (e.g., Aimovig).

In embodiments, the headache treatment is selected from a beta-blocker (e.g., atenolol, metoprolol, and nadolol); calcium channel blocker (e.g., diltiazem, nimodipine, and verapamil); an antidepressant (e.g., amitriptyline, paroxetine, and venlafaxine); and an anticonvulsant (e.g., gabapentin, pregabalin, and valproate). In embodiments, the headache treatment is a calcitonin gene-related peptide (CGRP) inhibitor, optionally selected from erenumab (e.g., AIMOVIG), fremanezumab (e.g., AJOVY), galcanezumab (e.g., EMGALITY), eptinezumab (e.g., VYEPTI), and rimegepant (e.g., NURTEC ODT).

In embodiments, the headache treatment is selected from divalproex sodium (e.g., DEPAKOTE), valproic acid, methysergide, propranolol, timolol, topiramate (e.g., TOPAMAX), and ubrogepant (e.g., UBRELVY). In embodiments, the headache treatment is ketamine. In embodiments, the migraine treatment is a ganglionic blocker (e.g., a depolarizing agent, a competitive agent (e.g., trimethaphan and tetraethylammonium), and a non-competitive agent (e.g., hexamethonium and mecamylamine).

In embodiments, the headache treatment includes transcranial stimulation. In embodiments, the transcranial stimulation is implantable, e.g., electrodes are implanted into certain brain areas. In embodiments, the headache treatment includes deep brain stimulation (DBS).

In embodiments, the present disclosure relates to the treatment and/or diagnosis and/or selection of treatment and/or monitoring of treatment response in connection with headache. In embodiments, headache includes, for instance, a migraine disorder, e.g., migraine with or without aura; hemiplegic migraine; cluster headaches; migrainous neuralgia; chronic headaches; tension headaches; headaches resulting from other medical conditions (e.g., infection or increased pressure in the skull due to a tumor); chronic paroxysmal hemicrania; miscellaneous headache unassociated with a structural lesion; headache associated with a non-vascular intracranial disorder; headache associated with the administration of a substance or its withdrawal; headache associated with a metabolic disorder; headache associated with a disorder of the cranium, neck, eyes, ears, nose, sinuses, teeth, mouth or other facial or cranial structure; cranial neuralgias; and nerve trunk pain and deafferentiation pain.

In embodiments, the headache is a migraine disorder. Migraine is a complex neurological condition that may be characterized by severe, episodic attacks of headache and associated features, which may include nausea, vomiting, sensitivity to light, sound or movement. In embodiments, the present methods reduce, prevent or alleviate nausea, vomiting, sensitivity to light, sound or movement. In some patients, the headache is preceded or accompanied by an aura.

In embodiments, the headache is or comprises a migraine disorder or headache.

In embodiments, the headache is diagnosed according to the third edition of the International Classification of Headache Disorders (ICHD-3).

In embodiments, the headache is or comprises a migraine headache. In embodiments, the headache is or comprises a chronic post-traumatic migraine headache. In embodiments, the headache is or comprises a persistent post-traumatic headache. In embodiments, the headache is or comprises a cluster headache. In embodiments, the headache is or comprises a tension headache. In embodiments, the headache is or comprises a sinus headache. In embodiments, the headache is or comprises a migraine headache. In embodiments, the headache is or comprises a hypnic headache. In embodiments, the headache is a chronic daily headache. In embodiments, the headache is a migraine headache, cluster headache, hemicrania continua headache, chronic headache, tension headache or chronic tension headache.

In embodiments, the migraine disorder is or comprises one or more symptoms selected from headaches, nausea, vomiting, difficulty speaking, numbness, tingling, and sensitivity to light and sound.

In embodiments, the migraine disorder is diagnosed or diagnosable by one or more of ID-Migraine, MIGSEV, the Visual Aura Rating Scale (VARS), Global Assessment of Migraine Severity (GAMS), Beck Depression Inventory (BDI), 9-item Patient Health Questionnaire (PHQ-9), Hospital Anxiety and Depression Scale (HADS), Migraine Disability Assessment Questionnaire (MIDAS), Migraine-Specific Quality of Life Survey (MSQ 2.1), European Quality of Life-Five Dimensions (EQ-5D), and Short-Form 36 (SF-36).

In embodiments, the migraine disorder is diagnosed or diagnosable by a visual analog scale (VAS). In embodiments, VAS scores are based on self-reported measures of symptoms that are recorded with a single handwritten mark placed at one point along the length of a 10-cm line that represents a continuum between the two ends of the scale-“no pain” on the left end (0 cm) of the scale and the “worst pain” on the right end of the scale (10 cm). 10 Measurements from the starting point (left end) of the scale to the patients' marks are recorded in centimeters and are interpreted as their pain.

In embodiments, the patient is experiencing symptoms associated with the prodrome stage of migraine. In embodiments, the patient is experiencing symptoms associated with the aura stage of migraine. In embodiments, the patient is experiencing symptoms associated with the attack stage of migraine. In embodiments, the patient is experiencing symptoms associated with the postdrome stage of migraine.

In embodiments, the headaches are or comprise headaches not primarily associated with disorders of the facial and cranial nerves.

In embodiments, the headaches are or comprise headaches primarily associated with disorders of the facial and cranial nerves.

In embodiments, the headaches not primarily associated with disorders of the facial and cranial nerves are selected from migraine, tension headache, meningeal irritation, brain tumor, temporal arteritis, and traumatic headache.

In embodiments, the headaches primarily associated with disorders of the facial and cranial nerves are selected from trigeminal neuralgia, atypical facial neuralgia, orbitally related neuralgias, Tolosa-Hunt syndrome, reader's paratrigeminal syndrome, postzoster syndrome, Costen's syndrome.

In embodiments, there is provided methods of treatment of headache and/or selection of agents for treatment of headache and/or monitoring if agents are having efficacy in treatment of headache. In embodiments, the treatment includes one or more of lessening severity, alleviation of pain intensity, and other associated symptoms, reducing frequency of recurrence, increasing the quality of life, and decreasing dose and/or frequency of administration of medications to treat the headache. For migraine, in embodiments, symptoms such as nausea, vomiting, and sensitivity to light, sound, and/or movement are treated. For cluster headache, in embodiments, symptoms such as swelling under or around the eyes, excessive tears, red eye, Rhinorrhea or nasal congestion, and red flushed face are treated.

In embodiments, there is provided methods of reducing incidence of headache and/or selecting agents to reduce incidence of headache. In embodiments, reducing incidence includes reducing one or more of severity (e.g., reducing need for and/or amount of therapies used in treatment), duration, and frequency (including, e.g., delaying or increasing time to next episodic attack in a subject).

In embodiments, there is provided methods of ameliorating headache or one or more symptoms of headache and/or selection of agents for ameliorating headache or one or more symptoms of headache and/or monitoring if agents are having efficacy in ameliorating headache or one or more symptoms of headache. In embodiments, ameliorating headache or one or more symptoms of headache includes lessening or improvement of one or more symptoms of headache as compared to without treatment. In embodiments, ameliorating headache or one or more symptoms of headache includes shortening or reduction in duration of a symptom. For example, in embodiments, headache is ameliorated by at least about 10%, or least about 20%, or least about 30%, or least about 40%, or least about 50%, or least about 60%, or least about 70% in the individual as compared to the level before treatment.

In embodiments, there is provided methods of controlling headache and/or selection of agents for controlling headache and/or monitoring if agents are having efficacy controlling headache. In embodiments, controlling headache includes maintaining or reducing severity or duration of one or more symptoms of headache or frequency of headache attacks in an individual (as compared to the level before treatment). For example, in embodiments, a duration or severity of head pain, or frequency of attacks is reduced by at least about 10%, or least about 20%, or least about 30%, or least about 40%, or least about 50%, or least about 60%, or least about 70% in the individual as compared to the level before treatment.

In embodiments, there is provided methods of delaying the development of headache or one or more symptoms of headache and/or selection of agents having efficacy in delaying the development of headache or one or more symptoms of headache and/or monitoring if agents are having efficacy in delaying the development of headache or one or more symptoms of headache. In embodiments, delaying the development of headache or one or more symptoms of headache includes hindering, slowing, stabilizing, and/or postponing progression of the disease. This delay can be of varying lengths of time, depending on the history of the disease and/or individuals being treated.

In embodiments, assessment of headache may be performed based on one or more subjective measures, such as patient characterization of symptoms, e.g., episodic attacks of headache lasting about 4 to about 72 hours with two or more of the following symptoms: unilateral pain, throbbing, aggravation on movement, and pain of moderate or severe intensity; and one or more of the following symptoms nausea or vomiting, and photophobia or phonophobia.

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one.

The term “alleviating” means a reduction in the occurrence of a pain, of a headache.

The term “treating” means to alleviate (or to eliminate) at least one symptom of pain (such as a headache pain), either temporarily or permanently.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

This invention is further illustrated by the following non-limiting examples.

### EXAMPLES

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1-Measuring Blink Reflex Response in Patients with and without Headache or Migraine and in Patients Before and After Undergoing Different Treatments

In this study, a population of male and female migraine patients are assessed for blink reflex response using the blink reflex measuring device. The patients in this study are tested when they visit the doctor during their routine office visits and/or before and after a doctor prescribed therapeutic intervention.

Preliminary results show significant differences between patients without a migraine and patients with a migraine as well as between those undergoing various treatments.

Blink reflex measuring device was used for collecting multiple blink-associated parameters from patients with headache/migraine before and after treatment undergoing treatment. The results are shown in TABLE 1.

TABLE 1
Pre & Post Treatment Trial Data

| | PRE-TX | POST-RX | PERCENT CHANGE | |
|---|---|---|---|---|
| VARIABLE | AVERAGE | AVERAGE | PRE-TO-POST TX | p VALUE |
| Latency (ms) | 47.953 | 50.231 | 5.00 | 0.052 |
| Differential Latency (ms) | 5.180 | 5.830 | 13.00 | 0.145 |
| Delta 30 (ms) | 5.046 | 6.156 | 22.00 | 0.026 |
| Initial Velocity (ms) | 4.532 | 4.368 | −4.00 | 0.308 |
| Time to Close (ms) | 31.352 | 30.600 | −3.00 | 0.140 |
| Time to Open (ms) | 303.450 | 246.098 | −19.00 | 0.069 |
| Time Under 20 (Ln ms) | 5.145 | 5.076 | −1.00 | 0.302 |
| Oscillations (no.) | 14.541 | 11.565 | −20.00 | 0.025 |
| Time to First Oscillation (Ln | 6.846 | 7.204 | 43.2 | 0.004 |
| ms) | | | | |
| Blinks (qty) | 6.538 | 5.965 | −9.00 | 0.296 |
| Blink Rate (per min) | 98.007 | 86.850 | −11.00 | 0.211 |
| Area Under Curve (pixels) | 36332.496 | 28648.679 | −21.00 | 0.059 |
| Max Closing Velocity (ms) | 8.417 | 8.210 | −2.00 | 0.396 |
| Max Opening Velocity (ms) | 2.978 | 2.709 | −9.00 | 0.085 |
| Excursion (pixels) | 124.891 | 116.957 | −6.00 | 0.185 |

A visual analog scale (VAS) was used to assess pain scores from patients before and after undergoing all treatment modalities for headache/migraine pain. The results are shown in FIG. 2.

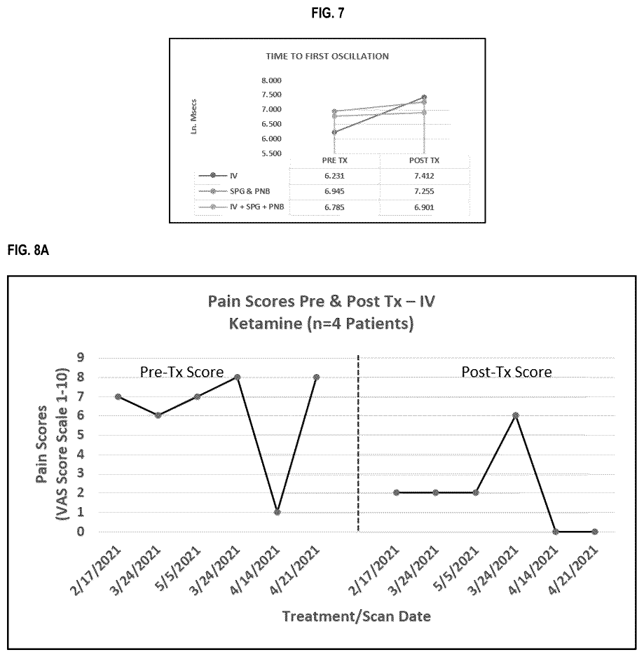

Blink reflex measuring device was used for collecting multiple blink-associated parameters from patients with headache/migraine before and after undergoing different treatments. The different treatments were intravenous (IV) ketamine infusion therapy, sphenopalatine ganglion (SPG) block+peripheral nerve block (PNB), and IV ketamine+SPG+PNB. The results are shown in TABLE 2, FIGS. 34-6D and FIG. 7.

TABLE 2
Pre & Post Trial by Treatment Modality Data

| | | PRE-TX | POST-TX | |
|---|---|---|---|---|
| VARIABLE | TX-MODALITY | AVERAGE | AVERAGE | p VALUE |
| Latency (ms) | IV | 46.518 | 49.827 | 0.219 |
| | SPG + PNB | 48.817 | 49.081 | 0.446 |
| | IV + SPG + PNB | 47.130 | 52.895 | 0.008 |
| Differential Latency (ms) | IV | 5.005 | 6.145 | 0.072 |
| | SPG + PNG | 5.476 | 5.562 | 0.424 |
| | IV + SPG + PNB | 5.462 | 6.012 | 0.269 |
| Delta 30 (ms) | IV | 4.433 | 7.435 | 0.006 |
| | SPG + PNB | 5.659 | 4.865 | 0.083 |
| | IV + SPG + PNB | 5.268 | 6.425 | 0.479 |
| Initial Velocity (ms) | IV | 4.092 | 3.200 | 0.078 |
| | SPG + PNB | 4.581 | 4.806 | 0.304 |
| | IV + SPG + PNB | 5.055 | 5.088 | 0.307 |
| Time to Close (ms) | IV | 32.282 | 29.930 | 0.181 |
| | SPG + PNB | 30.721 | 30.106 | 0.209 |
| | IV + SPG + PNB | 31.470 | 31.947 | 0.359 |
| Time to Open (ms) | IV | 368.125 | 307.108 | 0.403 |
| | SPG + PNB | 286.263 | 245.579 | 0.255 |
| | IV + SPG + PNB | 296.772 | 188.442 | 0.119 |
| Time Under 20 (Ln ms) | IV | 5.257 | 5.237 | 0.393 |
| | SPG + PNB | 5.185 | 5.057 | 0.348 |
| | IV + SPG + PNB | 5.062 | 4.794 | 0.209 |
| Oscillations (no.) | IV | 13.667 | 10.273 | 0.026 |
| | SPG + PNB | 14.030 | 12.757 | 0.191 |
| | IV + SPG + PNB | 16.558 | 12.478 | 0.243 |
| Time to First Oscillation (Ln | IV | 6.231 | 7.412 | 0.005 |
| ms) | SPG + PNB | 6.945 | 7.255 | 0.060 |
| | IV + SPG + PNB | 6.785 | 6.901 | 0.463 |
| Blinks (qty) | IV | 5.912 | 5.345 | 0.010 |
| | SPG + PNB | 6.388 | 6.361 | 0.435 |
| | IV + SPG + PNB | 8.172 | 6.915 | 0.457 |
| Blink Rate (per min) | IV | 95.603 | 83.490 | 0.010 |
| | SPG + PNB | 94.653 | 87.112 | 0.392 |
| | IV + SPG + PNB | 115.798 | 104.105 | 0.467 |
| Area Under Curve (pixels) | IV | 43881.648 | 29522.632 | 0.271 |
| | SPG + PNB | 33856.315 | 29979.208 | 0.315 |
| | IV + SPG + PNB | 36616.218 | 27034.005 | 0.225 |
| Max Closing Velocity (ms) | IV | 6.913 | 5.693 | 0.177 |
| | SPG + PNB | 8.670 | 8.962 | 0.382 |
| | IV + SPG + PNB | 9.797 | 10.380 | 0.281 |
| Max Opening Velocity (ms) | IV | 3.307 | 1.985 | 0.041 |
| | SPG + PNB | 2.848 | 2.950 | 0.429 |
| | IV + SPG + PNB | 2.778 | 3.070 | 0.174 |
| Excursion (pixels) | IV | 117.188 | 88.575 | 0.120 |
| | SPG + PNB | 122.983 | 125.13 | 0.411 |
| | IV + SPG + PNB | 137.555 | 138.707 | 0.225 |

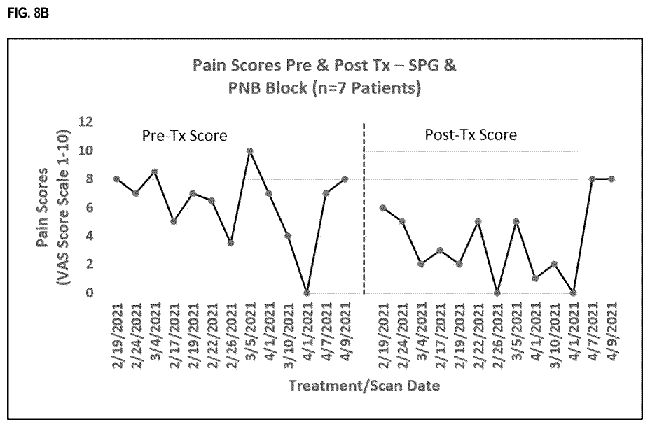

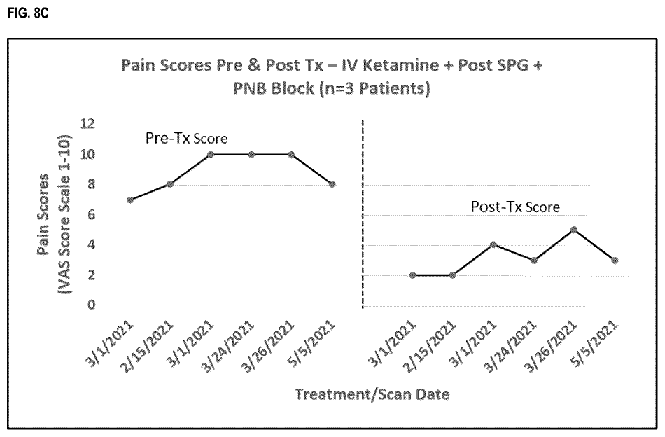

A visual analog scale (VAS) was used to assess pain scores from patients before and after undergoing each treatment modalities for headache/migraine pain. The different treatments were intravenous (IV) ketamine infusion therapy, sphenopalatine ganglion (SPG) block+peripheral nerve block (PNB), and IV ketamine+SPG+PNB. The results are shown in FIG. 8A-8C.

Blink reflex measuring device was used for collecting multiple blink-associated parameters from patients with headache/migraine vs. non-migraine controls. The results are shown in TABLE 3.

TABLE 3
Non-Migraine Patient Reflex Variables
vs. Migraine Patient Reflex Variables

| | PRE-TX | NON- | |
|---|---|---|---|
| | MIGRAINE | MIGRAINE | p |
| VARIABLE | AVERAGE* | CONTROLS | VALUE |
| Latency (ms) | 53.810 | 54.831 | 0.438 |
| Differential Latency (ms) | 5.573 | 7.169 | 0.138 |
| Delta 30 (ms) | 4.928 | 9.570 | 0.020 |
| Initial Velocity (ms) | 4.540 | 3.716 | 0.147 |
| Time to Close (ms) | 35.323 | 28.941 | 0.004 |
| Time to Open (ms) | 184.203 | 212.261 | 0.292 |
| Time Under 20 (Ln ms) | 4.726 | 4.716 | 0.444 |
| Oscillations (no.) | 13.553 | 9.016 | 0.099 |
| Blinks (qty) | 5.210 | 3.973 | 0.258 |
| Blink Rate (per min) | 86.075 | 66.350 | 0.261 |
| Area Under Curve (Ln pixels) | 10.061 | 9.875 | 0.319 |
| Max Closing Velocity (ms) | 9.388 | 7.017 | 0.127 |
| Max Opening Velocity (ms) | 3.735 | 1.870 | 0.009 |
| Excursion (pixels) | 140.670 | 100.054 | 0.033 |

*Age Range 56-67
N = 4

TABLES 4-6 show the variable changes based on right and left eye by treatment modality: intravenous (IV) ketamine infusion therapy (TABLE 4); sphenopalatine ganglion (SPG) block+peripheral nerve block (PNB) (TABLE 5); and IV ketamine+SPG+PNB (TABLE 6). It is important to note that whereas FIGS. 34-6D and FIG. 7 show the changes based on treatment modality of each variable and show the overall changes predicated on both right and left eye, TABLES 4-6 break down the variables by right and left eye changes.

TABLE 4
Pre & Post Treatment Modality Data with intravenous
(IV) ketamine infusion therapy

| | Left | Left | Right | Right |
|---|---|---|---|---|
| | Eye | Eye | Eye | Eye |
| Variable | Pre TX | Post Tx | Pre Tx | Post Tx |
| Latency (ms) | 45.888 | 53.958 | 46.266 | 52.583 |
| Differential Latency (ms) | 3.566 | 4.625 | 3.332 | 5.438 |
| Delta 30 (ms) | 3.507 | 9.328 | 2.983 | 5.766 |
| Initial Velocity (ms) | 4.989 | 3.601 | 4.941 | 3.269 |
| Time to Close (ms) | 34.940 | 27.686 | 33.001 | 31.119 |
| Time to Open (ms) | 492.117 | 286.813 | 361.481 | 258.318 |
| Time Under 20 (Ln ms) | 5.394 | 5.228 | 5.223 | 5.099 |
| Oscillations (no.) | 15.152 | 9.914 | 15.270 | 8.606 |
| Oscillations Rate (per min) | 3.991 | 2.293 | 4.056 | 1.966 |
| Time to First Oscillation | 6.157 | 7.301 | 6.310 | 7.517 |
| (Ln ms) | | | | |
| Blinks (qty) | 5.680 | 5.945 | 6.018 | 5.170 |
| Blink Rate (per min) | 90.576 | 89.415 | 95.844 | 73.691 |
| Area Under the Curve (pixels) | 11.046 | 10.250 | 10.742 | 10.157 |
| Max Closing Velocity (ms) | 8.370 | 6.281 | 8.427 | 5.759 |
| Max Opening Velocity (ms) | 2.937 | 2.253 | 3.089 | 1.834 |
| Excursions (pixels) | 149.324 | 91.548 | 139.732 | 92.605 |

TABLE 5
Pre & Post Treatment Modality Data with sphenopalatine
ganglion (SPG) block + peripheral nerve block (PNB)

| | Left | Left | Right | Right |
|---|---|---|---|---|
| | Eye | Eye | Eye | Eye |
| Variable | Pre Tx | Post Tx | Pre Tx | Post Tx |
| Latency (ms) | 50.987 | 52.630 | 49.465 | 53.687 |
| Differential Latency (ms) | 3.636 | 5.426 | 5.95S | 4.917 |
| Delta 30 (ms) | 4.377 | 5.488 | 4.832 | 5.081 |
| Initial Velocity (ms) | 4.815 | 4.914 | 4.739 | 5.066 |
| Time to Close (ms) | 31.489 | 33.223 | 33.135 | 31.553 |
| Time to Open (ms) | 335.786 | 274.913 | 303.411 | 272.996 |
| Time Under 20 (Ln ms) | 151.188 | 169.348 | 154.139 | 171.742 |
| Oscillations (no.) | 15.497 | 12.338 | 16.266 | 12.059 |
| Oscillations Rate (per min) | 3.670 | 2.941 | 3.927 | 2.913 |
| Time to First Oscillation (Ln ms) | 6.907 | 6.913 | 6.662 | 6.893 |
| Blinks (qty) | 7.212 | 5.336 | 8.196 | 7.243 |
| Blink Rate (per min) | 106.927 | 81.289 | 118.517 | 103.953 |
| Area Under the Curve (pixels) | 10.558 | 10.450 | 10.575 | 10.463 |
| Max Closing Velocity (ms) | 9.030 | 9.937 | 9.035 | 9.851 |
| Max Opening Velocity (ms) | 2.692 | 2.718 | 2.774 | 2.562 |
| Excursions (pixels) | 131.786 | 138.032 | 135.773 | 137.746 |

TABLE 6
Pre & Post Treatment Modality Data with IV ketamine + SPG + PNB

| | Left Eye | Left Eye | Right Eye | Right Eye |
|---|---|---|---|---|
| Variable | Pre Tx | Post Tx | Pre Tx | Post Eye |
| Latency (ms) | 49.207 | 50.834 | 47.915 | 48.459 |
| Differential Latency (ms) | 3.218 | 4.661 | 6.040 | 7.161 |
| Delta 30 (ms) | 3.881 | 4.414 | 5.908 | 6.224 |
| Initial Velocity (ms) | 4.610 | 4.739 | 4.779 | 4.586 |
| Time to Close (ms) | 30.756 | 30.739 | 31.667 | 31.356 |
| Time to Open (ms) | 296.863 | 221.115 | 241.055 | 237.510 |
| Time Under 20 (Ln ms) | 144.676 | 141.621 | 143.802 | 147.095 |
| Oscillations (no.) | 13.324 | 12.692 | 13.388 | 13.424 |
| Oscillations Rate (per min) | 3.420 | 2.923 | 3.410 | 3.054 |
| Time to First Oscillation | 6.940 | 7.290 | 6.947 | 7.221 |
| (Ln ms) | | | | |
| Blinks (qty) | 6.219 | 5.994 | 7.117 | 6.419 |
| Blink Rate (per min) | 95.273 | 82.312 | 111.017 | 89.515 |
| Area Under the Curve (pixels) | 33305.525 | 27504.063 | 29546.355 | 28607.354 |
| Max Closing Velocity (ms) | 8.830 | 8.602 | 8.987 | 8.774 |
| Max Opening Velocity (ms) | 3.240 | 3.383 | 3.238 | 2.836 |
| Excursions (pixels) | 123.563 | 125.028 | 127.198 | 124.294 |

### Summary from Results

Statistical differences (p=0.05) via Blink Reflex Measuring Device between patients having a migraine pre and post treatment.
Result from Blink Reflex Measuring Device correlates with Patient Pain Scale (VAS) Pre and Post treatment assessment.
Statistical differences (p=0.05) via Blink Reflex Measuring Device in patients with IV Ketamine treatment modality having a migraine pre and post treatment.
Blink Reflex Measuring Device could objectively identify the proper treatment for a patient.
Blink Reflex Measuring Device may be able to identify migraines before they happen.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of diagnosing whether a patient is afflicted with a headache, comprising:
(a) measuring a blink reflex in a patient suspected to be afflicted with a headache, the measurement comprising:
(i) applying a non-electrical stimulus to an eye of the patient to induce a blink reflex and
(ii) assessing one or more blink-associated parameters; and
(b) diagnosing the patient as afflicted with a headache or not afflicted with a headache by comparing the one or more blink-associated parameters to equivalent one or more blink-associated parameters from a patient who is not afflicted with a headache.

2. A method of predicting the onset of a headache in a patient afflicted with a headache, comprising:
(a) measuring a blink reflex in the patient, the measurement comprising:
(i) applying a non-electrical stimulus to an eye of the patient to induce a blink reflex and
(ii) assessing one or more blink-associated parameters; and
(b) determining if the patient is likely to experience an onset of a headache by comparing the one or more blink-associated parameters to equivalent one or more blink-associated parameters from a patient who is not afflicted with a headache and/or the patient when not experiencing symptoms of a headache or experiencing the onset of a headache.

3. A method of diagnosing and treating a patient afflicted with a headache, comprising:
(a) measuring a blink reflex in a patient suspected to be afflicted with a headache, the measurement comprising:
(i) applying a non-electrical stimulus to an eye of the patient to induce a blink reflex and
(ii) assessing one or more blink-associated parameters; and
(b) diagnosing the patient as afflicted with a headache or not afflicted with a headache by comparing the one or more blink-associated parameters to equivalent one or more blink-associated parameters from a patient who is not afflicted with a headache; and
(c) administering an effective amount of a headache treatment to a patient diagnosed as afflicted with a headache.

4. A method of selecting a headache treatment for a patient afflicted with a headache, comprising:
(a) measuring a blink reflex in the patient afflicted with a headache, the measurement comprising:
(i) applying a non-electrical stimulus to an eye of the patient to induce a blink reflex and
(ii) assessing one or more blink-associated parameters; and
(b) selecting a headache treatment for the patient afflicted with a headache by comparing the one or more blink-associated parameters to equivalent one or more blink-associated parameters from the patient when not experiencing symptoms of a headache and/or the patient when receiving the headache treatment.

5. A method of monitoring response of a headache in patient to a headache treatment, comprising
(a) measuring a blink reflex in the patient afflicted with a headache and who has undergone or is undergoing treatment with the headache treatment, the measurement comprising:
(i) applying a non-electrical stimulus to an eye of the patient to induce a blink reflex and
(ii) assessing one or more blink-associated parameters; and
(b) determining a response to the headache treatment by comparing the one or more blink-associated parameters to equivalent one or more blink-associated parameters from the patient when not experiencing symptoms of a headache and/or the patient before receiving the headache treatment.

6. The method of any one of claims 1-5, wherein the diagnosing comprises:
diagnosis of disease onset;
determination of disease stage and/or severity;
determination of disease progress; and/or
determination of cognitive state and/or mental capacity.

7. The method of any one of claims 1-6, wherein the method is carried out remotely, optionally with a telehealth device.

8. The method of any one of claims 1-7, wherein the method is adapted for point-of-care application.

9. The method of any one of claims 1-8, wherein an information collected can predict correlation of ADHD to the occurrence of a future disease and/or disorder of the central nervous system.

10. The method of any one of claims 1-9, wherein an information collected further allows for assessing the therapeutic efficacy of pharmaceutical and/or medical and/or surgical treatment modalities.

11. The method of any one of claims 1-10, wherein the method measures latency until physiological response to stimulus.

12. The method of any one of claims 1-11, wherein the blink reflex correlates with functionality of one or more of the trigeminal and facial nerves.

13. The method of any one of claims 1-12, wherein the non-electrical stimulus is one or more physical stimuli.

14. The method of any one of claims 1-13, wherein the one or more physical stimuli are a puff of air, a flash of light, a noise, and/or a vibration.

15. The method of any one of claims 1-14, wherein the air is or comprises compressed air.

16. The method of any one of claims 1-15, wherein the air is or comprises carbon dioxide.

17. The method of any one of claims 1-16, wherein the compressed air is applied across the eye.

18. The method of any one of claims 1-17, wherein the non-electrical stimulus is applied to the canthus or outer canthus.

19. The method of any one of claims 1-18, wherein the blink reflex is measured with a high-speed camera.

20. The method of any one of claims 1-19, wherein the blink reflex is not measured with an EMG sensor.

21. The method of any one of claims 1-20, wherein the blink reflex is measured with a blink reflex monitoring device.

22. The method of any one of claims 1-21, wherein the blink reflex monitoring device is a wearable device or a handheld device or can be placed on a tripod, on a desk, or the like.

23. The method of any one of claims 1-22, wherein the blink reflex monitoring device is a wearable device.

24. The method of any one of claims 1-23, wherein the blink reflex monitoring device has or comprises:
a front end, a back end, a first plane extending through the front end and the back end,
a second plane intersecting the first plane between the front end and the back end, the back end configured to receive a portion of a face of a user and disposed opposite the front end; and
a strap coupled to the blink reflex monitoring device and having a first position and a second position,
wherein in the first position the strap extends away from the blink reflex monitoring device along the first plane end and is configured to secure the blink reflex monitoring device to a head of a user and in the second position the strap extends below the blink reflex monitoring device along the second plane and is configured to support the blink reflex monitoring device above a surface.

25. The method of any one of claims 1-24, wherein the blink reflex monitoring device has or comprises:
a front end and a back end, the back end configured to receive a portion of a face of a user and disposed opposite the front end;
at least one strap coupled to the blink reflex monitoring device proximate the back end, the at least one strap configured to be secured around a head of a user; and
an air compression system including a source of compressed air and a valve, the air compression system disposed within the blink reflex monitoring device and the valve configured to output compressed air from the back end at a pressure of about 3 PSI to about 60 PSI.

26. The method of any one of claims 1-25, wherein the air compression system disposed within the blink reflex monitoring device and the valve configured to output compressed air from the back end at a pressure of about 30 PSI to about 50 PSI.

27. The method of any one of claims 1-26, wherein the blink reflex is not measured with an electromyography (EMG) device.

28. The method of any one of claims 1-27, wherein the blink reflex is measured with a high-speed camera.

29. The method of any one of claims 1-28, wherein the blink reflex is not measured with an EMG sensor.

30. The method of any one of claims 1-29, wherein the measuring of a blink reflex, comprises:
providing a blink reflex monitoring device to a user, the blink reflex monitoring device having an air compression system including a compressed air source and a valve;
outputting a first burst of compressed air from the compressed air source through the valve at a first pressure;
detecting a presence or an absence of a blink in response to the first burst of compressed air source at the first pressure;
if the blink is present, then measuring a duration between the output of the first burst of compressed air at the first pressure and the blink to determine the blink reflex; and
if the blink is absent, then outputting a second burst of compressed air from the compressed air source at a second pressure, the second pressure being greater than the first pressure and measuring a duration between the output of the second burst of compressed air at the second pressure and the blink to determine the blink reflex.

31. The method of any one of claims 1-30, wherein the one or more blink-associated parameters are selected from:
(a) latency, optionally in milliseconds, comprising a time differential between stimulation and eyelid movement, e.g., one or more of upper eyelid or lower eyelid;
(b) differential latency, optionally in milliseconds, comprising a time differential between the start of ipsilateral eye movement and the start of contralateral eye movement;
(c) delta 30, comprising a time difference between ipsilateral eye and contralateral eye movement;
(d) eyelid excursion, optionally in pixels, comprising a distance traveled by the eyelid from the tonic lid position to closed position;
(e) initial lid velocity, optionally in pixels/msec, comprising an average eyelid speed following start of eyelid movement, e.g., the first about 5 frames, the first about 7 frames, or the first about 10 frames;
(f) time to close, optionally in log scale, comprising a time for lid to travel from tonic lid position to the closed position;
(g) time to open, optionally in log scale, comprising a time for lid to travel from closed position back to tonic lid position;
(h) time under threshold, optionally in log scale, comprising a time that the eyelid spends below the threshold position;
(i) number of oscillations, comprising cycles of up and down upper eyelid movement after a stimulated blink;
(j) total blink time, optionally in log scale, comprising a time from start of eyelid movement until it returns to its tonic lid position;
(k) number of blinks;
(l) blink rate, optionally per minute;
(m) area under curve, optionally in pixels;
(n) maximum closing velocity, optionally in milliseconds, comprising a maximum velocity during eyelid closure;
(o) maximum opening velocity, optionally in milliseconds, comprising a maximum velocity during eyelid opening; and
(p) time to first oscillation, optionally in log scale, comprising the elapsed time between the eyelid returning within threshold of a tonic position for a stimulated blink and the onset of the next unstimulated blink.

32. The method of any one of claims 1-31, wherein in comparison of the patient before receiving the headache treatment to after receiving the headache treatment
latency increases;
differential latency increases;
delta 30 increases;
eyelid excursion decreases;
initial lid velocity decreases;
time to close decreases;
time to open decreases;
time under threshold decreases;
number of oscillations decreases;
total blink time decreases;
number of blinks decreases;
blink rate decreases;
area under curve decreases;
maximum closing velocity decreases;
maximum opening velocity decreases; and/or
time to first oscillation increases.

33. The method of any one of claims 1-32, wherein in comparison of a patient who is not afflicted with a headache with a patient who is afflicted with a headache:
latency decreases;
differential latency decreases;
delta 30 decreases;
eyelid excursion increases;
initial lid velocity increases;
time to close increases;
time to open decreases;
time under threshold increases;
number of oscillations increases;
total blink time decreases;
number of blinks increases;
blink rate increases;
area under curve increases;
maximum closing velocity increases; and/or
maximum opening velocity increases.

34. The method of any one of claims 1-33, wherein the headache treatment is selected from over-the-counter medications such as aspirin, ibuprofen (e.g., Advil and Motrin IB), and acetaminophen (e.g., Tylenol), prescription medications, such as triptans such as sumatriptan (e.g., Imitrex) and zolmitriptan (e.g., Zomig), and preventive medications such as metoprolol (e.g., Lopressor), propranolol (e.g., Innopran and Inderal), amitriptyline, divalproex (e.g., Depakote), topiramate (e.g., Qudexy XR, Trokendi XR, and Topamax), and erenumab-aooe (e.g., Aimovig).

35. The method of any one of claims 1-34, wherein the headache treatment is selected from a beta-blocker (e.g., atenolol, metoprolol, and nadolol); calcium channel blocker (e.g., diltiazem, nimodipine, and verapamil); an antidepressant (e.g., amitriptyline, paroxetine, and venlafaxine); and an anticonvulsant (e.g., gabapentin, pregabalin, and valproate).

36. The method of any one of claims 1-35, wherein the headache treatment is a calcitonin gene-related peptide (CGRP) inhibitor, optionally selected from erenumab (e.g., AIMOVIG), fremanezumab (e.g., AJOVY), galcanezumab (e.g., EMGALITY), eptinezumab (e.g., VYEPTI), and rimegepant (e.g., NURTEC ODT).

37. The method of any one of claims 1-33, wherein the headache treatment is selected from divalproex sodium (e.g., DEPAKOTE), valproic acid, methysergide, propranolol, timolol, topiramate (e.g., TOPAMAX), and ubrogepant (e.g., UBRELVY).

38. The method of any one of claims 1-37, wherein the headache treatment is ketamine.

39. The method of any one of claims 1-38, wherein the headache treatment is a ganglionic blocker (e.g., a depolarizing agent, a competitive agent (e.g., trimethaphan and tetraethylammonium), and a non-competitive agent (e.g., hexamethonium and mecamylamine).

40. The method of any one of claims 1-39 wherein the headache is or comprises a migraine disorder or headache and/or wherein the headache treatment is or comprises a migraine treatment.

41. The method of any one of claims 1-40, wherein the headache is diagnosed according to the third edition of the International Classification of Headache Disorders (ICHD-3).

42. The method of any one of claims 1-41, wherein the headache is or comprises a chronic post-traumatic migraine headache.

43. The method of any one of claims 1-42, wherein the headache is or comprises a persistent post-traumatic headache.

44. The method of any one of claims 1-43, wherein the headache is or comprises a cluster headache.

45. The method of any one of claims 1-44, wherein the headache is or comprises a tension headache.

46. The method of any one of claims 1-45, wherein the headache is or comprises a sinus headache.

47. The method of any one of claims 1-46, wherein the headache is or comprises a migraine disorder or headache.

48. The method of any one of claims 1-47, wherein the headache is or comprises a hypnic headache.

49. The method of any one of claims 1-48, wherein the headache is a chronic daily headache.

50. The method of any one of claims 1-49, wherein the headache is a migraine headache, cluster headache, hemicrania continua headache, chronic headache, tension headache or chronic tension headache.

51. The method of claim 1-50, wherein the patient is experiencing symptoms associated with the prodrome stage of migraine.

52. The method of claim 1-51, wherein the patient is experiencing symptoms associated with the aura stage of migraine.

53. The method of claim 1-52, wherein the patient is experiencing symptoms associated with the attack stage of migraine.

54. The method of claim 1-53, wherein the patient is experiencing symptoms associated with the postdrome stage of migraine.
